(19) United States
(12) Patent Application Publication
(10) Pub. No.: US 2024/0155984 A1
(43) Pub. Date: May 16, 2024

(54) APPARATUS FOR ORGANIC MATTER CULTIVATION

(71) Applicant: KURT MICHAEL SWANSON, Philadelphia, PA (US)

(72) Inventor: KURT MICHAEL SWANSON, Philadelphia, PA (US)

(21) Appl. No.: 18/507,073

(22) Filed: Nov. 12, 2023

Related U.S. Application Data
(60) Provisional application No. 63/383,385, filed on Nov. 11, 2022.

(51) Int. Cl. A01G 18/65; A01G 18/55; A01G 18/69; C12N 1/14

(52) U.S. Cl. CPC A01G 18/65; A01G 18/55; A01G 18/69; C12N 1/14

(57) ABSTRACT

A self-contained apparatus for organic matter cultivation is provided, the apparatus having a cultivation chamber, a lid associated with the cultivation chamber, a substrate arranged within the cultivation chamber, an access point associated with the lid, and at least one air exchange port associated with the lid.

### CROSS REFERENCE TO RELATED APPLICATION

This application claims the benefit of U.S. Provisional Application Ser. No. 63/383,385 filed Nov. 11, 2022, which is incorporated by reference as if fully set forth herein.

### FIELD OF INVENTION

This application is generally related to the cultivation of organic matter, and more particularly related to an apparatus with substrate for the sanitary cultivation of fungi such as mushrooms.

### BACKGROUND

Fungi cultivation at home, including the cultivation of medicinal and culinary mushrooms, has traditionally involved the use of grow kits, most commonly in the form of grow bags. These bags typically consist of a substrate that is infused with mushroom spores, contained within a plastic bag. While popular, these grow bags exhibit several shortcomings that limit their effectiveness and user appeal. Current grow bags offer minimal control over environmental factors critical to mushroom cultivation, such as humidity, temperature, and light exposure. This lack of control often leads to suboptimal growth conditions, affecting both the yield and quality of the mushrooms. The semi-open nature of grow bags increases the risk of contamination from mold, bacteria, and other fungi. Contamination not only jeopardizes the health of the mushrooms but can also pose a health risk to the cultivator. Grow bags typically require regular manual intervention, such as misting for humidity control and temperature monitoring. This can be time-consuming and may be challenging for users without prior experience in mushroom cultivation. Grow bags are often not designed with aesthetics in mind and can be visually unappealing. Additionally, they can occupy significant space, which is a constraint in urban settings and smaller homes.

Given these limitations and the growing popularity of home cultivation of mushrooms, there is a need for a more advanced and user friendly solution that provides a self-contained apparatus for growing mushrooms that reduces the risk of contamination and allows for easier cultivation. The present invention addresses the shortcomings of traditional grow kits by introducing a self-contained apparatus that provides an intuitive and efficient method of cultivating mushrooms at home.

### SUMMARY

A self-contained apparatus that may be sterilized and containing pre-sterilized substrate, a mechanical and/or chemical binding agent for ensuring suitability for shipping abuse, removable covers atop filter patch or patches for the cultivation of organic matter including fungi such as mushrooms.

A method of growing fungi using a self-contained apparatus, the method comprising of the steps of providing a self-contained apparatus with substrate, introducing mushroom inoculant through an injection port, introducing filtered air exchange to the inner chamber of the apparatus, replacing the lid of the apparatus with a removable dome to ensure room for mushroom growth and introduce water to ensure a humid growth environment.

For sake of brevity, this summary does not list all aspects of the present application, which are described in further detail below.

### BRIEF DESCRIPTION OF THE DRAWINGS

The foregoing summary, as well as the following detailed description of the preferred embodiments of the present application, will be better understood when read in conjunction with the appended drawings. For the purpose of illustrating the invention, there is shown in the drawings embodiments that are presently preferred. It should be understood, however, that the inventions are not limited to the precise arrangements shown in the drawings.

FIG. 1 is a front elevation view of an embodiment of an apparatus for organic matter cultivation;

FIG. 2 is another front elevation view of the apparatus shown in FIG. 1, demonstrating the use of a syringe to introduce organic materials into the apparatus for cultivation;

FIG. 3 is another front elevation view of the apparatus shown in FIG. 1, demonstrating the opening of a first patch cover to introduce filtered air exchange in the apparatus;

FIG. 4 is another front elevation view of the apparatus shown in FIG. 1, demonstrating the opening of a second patch cover to introduce additional filtered air exchange in the apparatus;

FIG. 5 is another front elevation view of the apparatus shown in FIG. 1, demonstrating the growth of organic matter and the use of a protection dome;

FIG. 6 is a top plan view of a cover label with removable sections associated with different ports of the apparatus shown in FIG. 1;

FIG. 7 is a front perspective view of the apparatus shown in FIG. 1, contained in a sealed bag;

FIG. 8 is another front perspective view of the apparatus shown in FIG. 1.

### DETAILED DESCRIPTION OF THE PREFERRED EMBODIMENTS

Certain terminology is used in the following description for convenience only and is not limiting. The words “front,” “back,” “top,” “bottom,” “inner,” “outer,” “upper,” “lower,” “internal,” and “external” designate directions in the drawings to which reference is made. The words “upward,” “downward,” “above,” and “below” refer to directions towards a higher or lower position from the parts referenced in the drawings. The words “inward” and “outward” refer to directions towards an inner or outer portion of the element referenced in the drawings. The words “clockwise” and “counterclockwise” are used to indicate opposite relative directions of rotation, and may be used to specifically refer to directions of rotation about an axis in accordance with the well-known right hand rule. Additionally, the terms “a” and “one” are defined as including one or more of the referenced item unless specifically noted otherwise. A reference to a list of items that are cited as “at least one of a, b, or c” (where a, b, and c represent the items being listed) means any single one of the items a, b, or c, or combinations thereof. The terminology includes the words specifically noted above, derivatives thereof, and words of similar import.

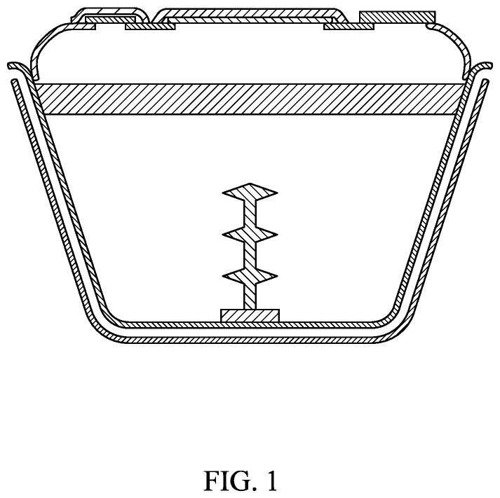

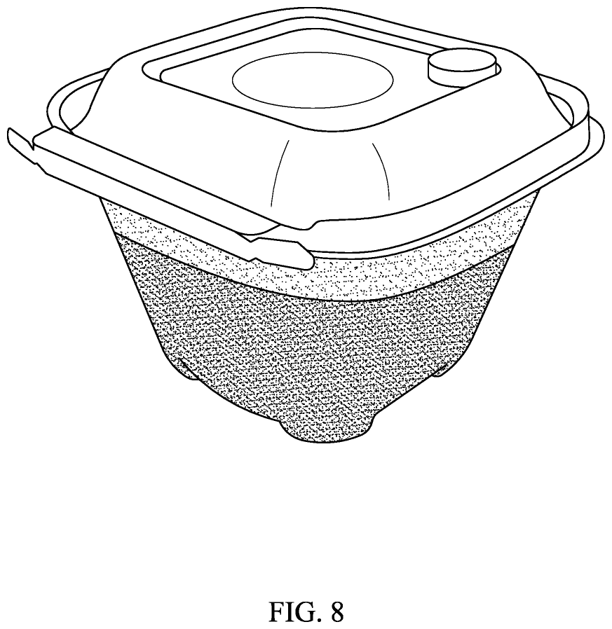

FIGS. 1-8 show an embodiment of an apparatus 20 for organic matter cultivation according to the present application, which may include a cultivation chamber 21 having an internal cavity 22 that is suited for holding substrate 23 for the cultivation of organic matter. The internal cavity 22 includes a base 24 and side walls 26, and an opening 28. The apparatus 20 further includes a top lid 30 that can be arranged to cover the opening of the internal cavity 22. The internal cavity 22 may further include a substrate support 29 associated with the base 24 of the internal cavity, which may specifically be configured to minimize movement of the substrate 23 within the internal cavity 22. Although the presence of a substrate support 29 is not necessary for the use of the apparatus 20 to cultivate organic matter, the substrate support 29 is helpful to keep the substrate 23 in place especially when the apparatus is being handled such as during shipping. The substrate support 29 may take a variety of different forms, including for example and within limitation in the shape of a screw as shown in FIGS. 1-5, a fin, a T-shape, a hook, a web, a mesh, one or more of the foregoing shapes, or a combination of these elements.

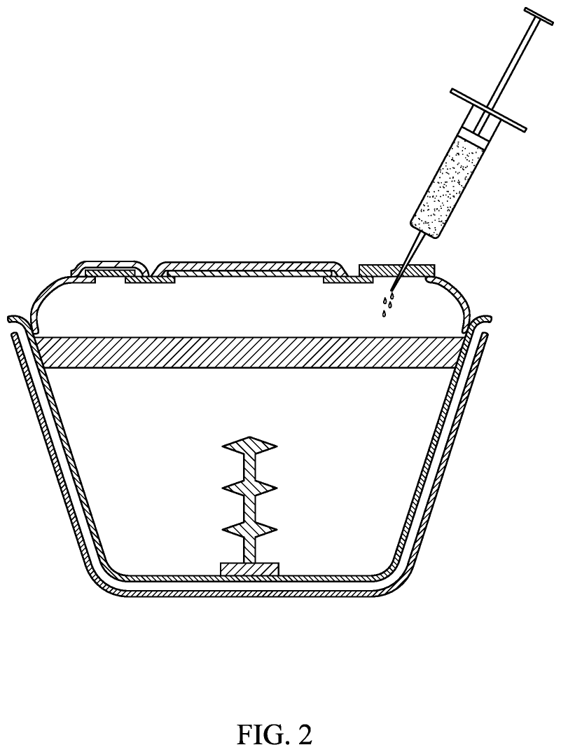

As further illustrated in FIG. 1, the top lid 30 may include one or more openings or ports 32, 34, 36, which may each be associated with removable or non-removable covers 42, 44, 46. The top lid 30 may be configured to tightly cover or seal the base chamber 21 such that there is minimal or no airflow to the cultivation chamber 21 once the top lid 30 is used to cover the opening of the internal cavity 22. This can be achieved through, for example and without limitation, a pressure fit, a snap fit, a screw top, or other means. The one or more openings or ports 32, 34, 36 of the top lid 30 may include at least one access point 32, which can be used to introduce organic matter into the apparatus 20 for cultivation. For example and without limitation, as shown in FIG. 2 the access point 32 can be used to inoculate the substrate 23 contained in the internal cavity 23 of the cultivation chamber 21 by inserting the needle of a syringe into the covered access point 32. The cover 42 associated with the access point 32 can be removable or non-removable, and can be formed from a material such as foam, plastic, or other appropriate materials that can be easily punctured by a needle but still maintain minimal airflow after the needle is removed.

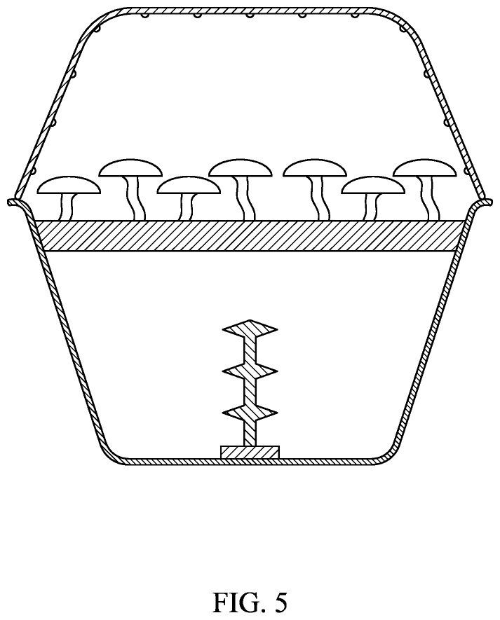

The one or more openings or ports 32, 34, 36 of the top lid 30 may further include at least one air exchange opening 34, and in certain embodiments at least a second larger air exchange opening. Each of the air exchange openings 34, 36 may be covered by an air exchange filter that may in turn be covered by a removable cover 44, 46 which, when peeled off, permit the progressive increase in free-air exchange to support the life cycle of mushroom spore or culture inoculation. The removable covers 44, 46 may be configured as patch covers that can be easily peeled off and then replaced to seal the air exchange filters of the air exchange openings 34, 36. The top lid 30 may be removed, and replaced with an attachable dome or protection dome 50 as shown in FIG. 5. The dome 50 helps maintain proper environmental conditions for mushroom growth. These components ensure a clean, sanitary environment for mushrooms to grow and prevent the contamination of the substrate, mycelium, and mushrooms by other organisms and microorganisms prior to successful harvesting.

The user receives the sealed container, and initially inoculates the container and its contained substrate via a self-healing injection port or re-sealable open hole with a syringe or other inoculation source of the desired mushroom genetics (spores or culture). Following the inoculation, the user peels off a film or sticker which covers a small air-exchange port which is covered by a filter patch. This allows a small amount of air exchange to support colonization but prevents the introduction of foreign spores or other microorganisms and insects in the user's environment.

After the initial colonization period, the user removes a film or sticker from a second larger port (also covered by a filter patch) which allows an increase in free-air exchange (increasing oxygen and reducing carbon dioxide) to the colonized substrate block which supports the development from mycelium to fruiting bodies (mushrooms). Following this development, the organism becomes resistant to contamination, and the container (which may have a tamper-evident seal) can be opened by the user to introduce moisture which is needed to ensure the proper growth of mushrooms from pins to larger fruit bodies. As the mushrooms develop in size, the dome which is included in the apparatus and may be nested to the main container, replaces the lid and provides space for the mushrooms to expand in size. This dome ensures a proper climate and keeps out microorganisms and insects. The user adds moisture via a spray bottle to the dome and substrate itself during this process and ultimately harvests the mushrooms after they mature.

The substrate may contain a chemical binder such as Agar, which ensures the substrate remains a mechanically solid block during transit and prevent crumbling because of physical abuse of the package during transit. This binder may also be selected as a nutritious material for mushroom consumption. The substrate container may have a retaining mechanism in the main body that protrudes into the substrate to ensure it does not shift in transit. Alternatively, it may have a spacer on top which prevents it from moving out of position in transit.

The substrate may contain an enriched addition of cereal grains or other nutritious spawn grains, such as rice, wheat berries, millet, or other to facilitate a quick initial colonization by the mushroom spores or culture that are introduced by the user. These spawn grains may form a top layer, or the top portion of substrate may be mix with the spawn grains, or the spawn grains may be mixed throughout the substrate block evenly. The substrate may vary based on the desired mushroom to be grown but may consist of mixtures or a single component of hardwood chips or powder, animal manure, coconut husks, straw, or other agricultural waste products as a source of nutrition for the mushrooms.

It is critical that the initial substrate and associated components be sterilized following production. The entire container may be sterilized via high pressure steam autoclave, and even be contained in a sterilization bag to ensure that the contents, apparatus itself, and outer surfaces of the container are free of any initial microbes which would spoil the cultivation for the user. This can prevent the need for the user to sterilize the external surfaces of the container, as they would already be sterile at the time of removal from the bag, thus eliminating the need to wipe the container with alcohol or sterilize the needle after withdrawing it from the injection port.

One who wishes to cultivate small amounts of mushrooms for personal use would use this apparatus and associated process to grow mushrooms simply and easily with limited need for specialized growing conditions or equipment.

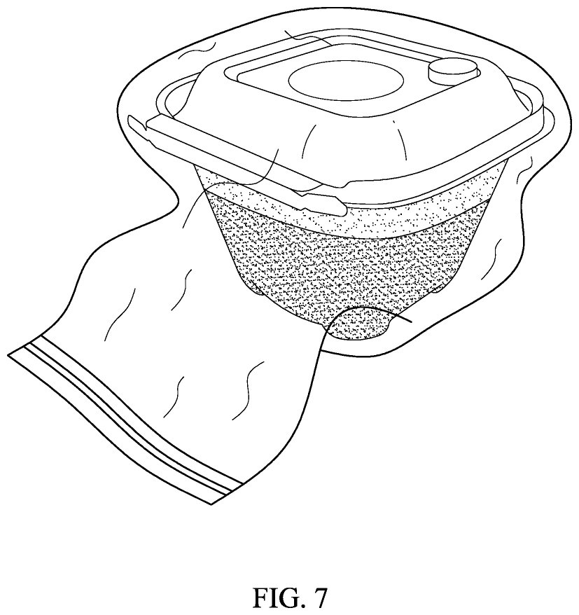

As further shown in FIG. 1, the self-contained apparatus can be delivered to a user already sterilized, containing a substrate block with a possible grain spawn layer. The substrate block may be associated with or retained to the cavity of the apparatus via a fastener or other mechanism or a chemical binding agent to reduce damage during shipment. Another method of mechanical restraint of the substrate block may include a spacer at the top of the substrate block. This entire apparatus may be contained within a sealed sterile bag and autoclaved to ensure all interior and external surfaces are sterile and contain to microbial contaminants, as shown in FIG. 7.

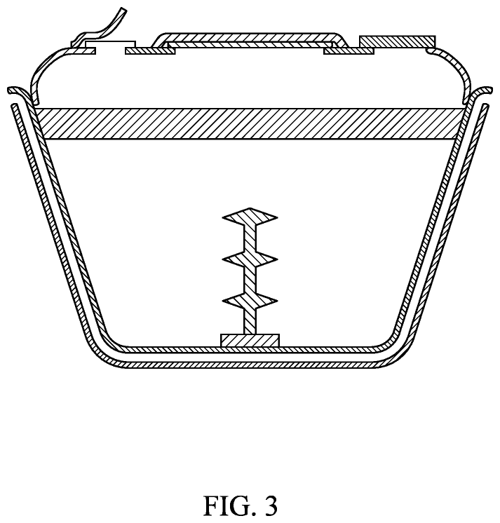

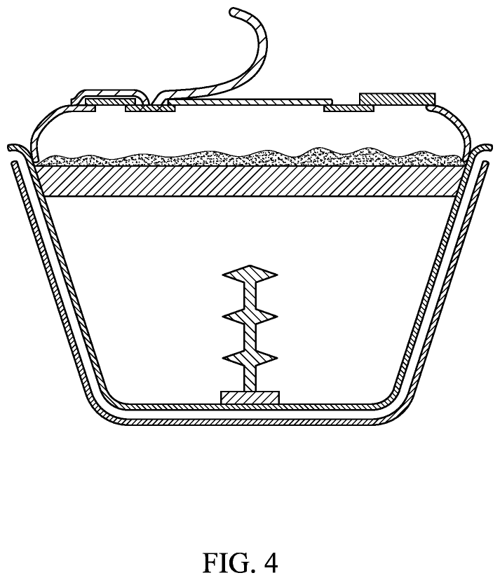

As shown in FIG. 2, the user may supply their own mushroom inoculant, such as liquid cultures or spores, and introduce the inoculant into the self-contained apparatus through an injection port via a syringe or similar mechanism. As shown in FIG. 3, the user can easily peel away the first air filter patch cover, introducing a small amount of filtered air exchange to support mycelial colonization of the spawn or substrate block. As shown in FIG. 4, following mycelial colonization, the user may remove a second air filter patch cover, introducing a larger amount of filtered free air exchange to support further growth at this stage of cultivation. As mushroom pins begin to grow, as illustrated in FIG. 5, the user can remove the lid and replace it with an included removable dome to ensure room for the mushrooms to grow in a sanitary environment that can be sprayed with water to ensure a humid growth environment throughout the process.

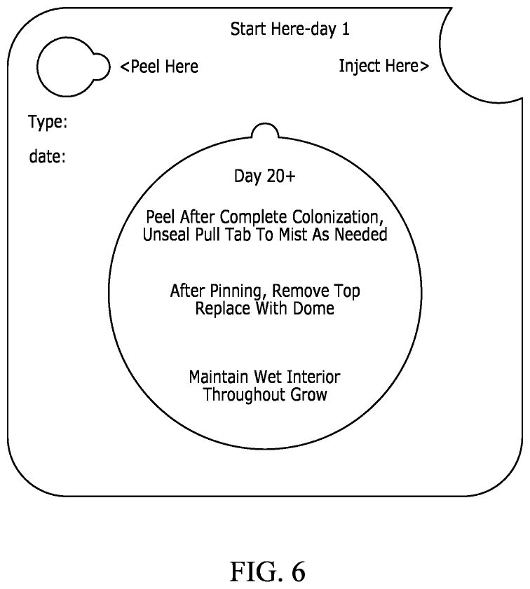

The entire growing process can be made more user-friendly by including instructions on the various ports and patches of the apparatus. For example, as shown in FIG. 6, a top cover sticker array with printed instructions may be placed on the top lid, having peelable sections that cover filtered fresh air ports. The stickers are arranged to cover the initial filtered free air exchange port, a larger port for later removal, and a cutout to allow for the adhesion of a self-healing port that covers the injection hole for introducing inoculant. The sticker array may include instructions for the user on the time periods for performing certain actions.

As shown in FIG. 7, when shipping the apparatus to a user, the complete autoclaved and sterilized apparatus and contained substrate can be provided inside of a sealed bag that is ready for use. After the substrate has been inoculated, the first filter port cover can be peeled open to introduce air flow. However, the second filter port cover is not removed before colonization is complete. The self-healing port may be a raised part, which can be penetrated with a mushroom spore syringe, but no further action is required to keep air or contaminants from entering the apparatus.

While various methods, configurations, and features of the present compact air conditioner have been described above and illustrated in the drawings, one of ordinary skill will appreciate from the disclosure that any combination of the above features can be used without departing from the scope of the present application. It is also recognized by those of ordinary skill in the art that many changes, only a few of which are exemplified in the detailed description above, may be made to the above described methods and embodiments without departing from the broad inventive concepts and principles embodied therein. The present embodiments are therefore to be considered in all respects as illustrative and not restrictive, the scope of the invention being indicated by the appended claims rather than by the foregoing description, and all changes which come within the meaning and range of equivalency of the claims are therefore to be embraced therein.

What is claimed is:

1. A self-contained apparatus for organic matter cultivation comprising:
a cultivation chamber;
a lid associated with the cultivation chamber;
a substrate arranged within the cultivation chamber;
an access point associated with the lid; and
at least one air exchange port associated with the lid.

2. The self-contained apparatus of claim 1, further comprising a removable dome that can be associated with the cultivation chamber after the lid is removed.

3. The self-contained apparatus of claim 1, further comprising a substrate support associated with the substrate and the cultivation chamber.

4. The self-contained apparatus of claim 1, wherein the access point is a self-healing injection port.

5. The self-contained apparatus of claim 1, wherein the access point is an opening with a resealable cover.

6. The self-contained apparatus of claim 1, further comprising a removable covering arranged over the at least one air exchange port.

7. A self-contained apparatus for cultivating fungi, the apparatus comprising:
a sterilized container;
a sterilized substrate material arranged within an internal cavity of the container; and
a removable lid that fits over an opening of the internal cavity of the container, the lid comprising an access point for inoculation, and a covered air exchange filter.

8. The self-contained apparatus of claim 7, wherein the covered air exchange filter is comprised of a peelable sticker arranged over the air exchange filter.

9. The self-contained apparatus of claim 8, further comprising a removable dome.

10. The self-contained apparatus of claim 9, further comprising a bag that encloses the apparatus.

11. A method of growing fungi, the method comprising the steps of:
providing a self-contained apparatus with a substrate material arranged within an internal cavity of the apparatus, and a lid associated with an opening of the internal cavity;
introducing an inoculant through an injection opening located on the lid;
introducing filtered air exchange through a covered air port located on the lid;
removing the lid;
placing a dome associated with an opening of the internal cavity.

12. The method of claim 11, wherein the step of introducing an inoculant further comprises injecting the inoculant using a syringe that pierces the injection opening located on the lid.

13. The method of claim 11, wherein the step of introducing filtered air exchange through a covered air port further comprises peeling off a cover that is arranged over the covered air port.

14. The method of claim 13, wherein the step of introducing filtered air exchange through a covered air port comprises peeling of a second cover that is arranged over a second air port.

15. The method of claim 11, further comprising the steps of removing the dome, introducing water into the internal cavity and substrate material, and placing the dome back over the opening of the internal cavity to maintain the humidity inside of the self-contained apparatus.
